(19) United States
(12) Patent Application Publication
(10) Pub. No.: US 2009/0149508 A1
(43) Pub. Date: Jun. 11, 2009

(54) Process for the preparation of substituted phenyl ether compounds

(75) Inventors: Johannes Ludescher, Breitenbach (AT); Rashid Abdul Rehman Khan, Maharashtra (IN); Aniruddha Paul, Maharashtra (IN)

Correspondence Address: SANDOZ INC, 506 CARNEFIE CENTER, PRINCETON, NJ, 08540, US

(73) Assignee: Sandoz AG, Basel (CH)

(21) Appl. No.: 11/989,252

(22) PCT Filed: Jul. 25, 2006
(86) PCT No.: PCT/EP2006/007315

§ 371 Date: Jan. 23, 2008

(30) Foreign Application Priority Data
Jul. 27, 2005 (SI) P200500218

(51) Int. Cl. A61K 31/44; C07D 213/72; A61P 3/10; C07D 417/00

(52) U.S. Cl. 514/342; 546/312; 546/268.7

(57) ABSTRACT

A novel process for the preparation of a compound of the formula (II), which is useful as intermediate compound for the preparation of thiazolidinedione derivatives, such as rosiglitazone, pioglitazone, troglitazone and ciglitazone, is disclosed.

### FIELD OF THE INVENTION

The present invention relates to a novel process for the preparation of substituted phenyl ether compounds which may be used as key intermediates for the preparation of thiazolidinedione derivatives, useful in the treatment of Type II diabetes.

More precisely, the present invention relates to a process for the preparation of certain pyridyl substituted ethoxy benzaldehydes (ether compounds) which may be used as intermediates for the synthesis of thiazolidinedione derivatives, such as rosiglitazone, pioglitazone, troglitazone and ciglitazone or a pharmaceutically acceptable acid addition salt thereof having hypoglycemic and hypolipidemic activities.

### BACKGROUND OF THE INVENTION

EP 0257781 B1 describes a process for the preparation of 4-[2-(5-ethylpyridyl)ethoxy]benzaldehyde which is used for the preparation of pioglitazone. The process described in this patent requires long reaction time and uncontrolled impurities in the desired compound are obtained.

EP 0506273 B1 describes a process for the preparation of 4-[2-(5-ethylpyridyl)ethoxy]benzaldehyde by reacting potassium salt of hydroxybenzaldehyde with 2-(5-ethylpyridyl)ethyl methanesulfonate. This process involves corrosive chemicals, such as p-toluenesulphonyl chloride and methanesulphonyl chloride, and an additional steps which limits the use in industrial process.

U.S. Pat. No. 6,100,403 describes another method for the preparation of the said intermediate which involves using a mixture of toluene and ethanol as reaction solvents and potassium carbonate as a base. However this process limits the recovery of solvents.

EP 306228 B1 describes the coupling reaction of 2-(N-methyl-N-(2-pyridyl)amino)ethanol with 4-fluorobenzaldehyde in the presence of N,N-dimethylformamide (DMF) as a solvent and sodium hydride as a base to obtain 4-[2-(N-methyl-N-(2-pyridyl)amino)ethoxy]benzaldehyde, which is the key intermediate compound in the preparation of rosiglitazone. The said process involves hazardous, expensive chemicals such as sodium hydride and poor yield of product limits the use it in commerciale scale batches.

Cantello et al. (J. Med. Chem., Vol. 37, No. 23, 1994, pp. 3977-3985) have prepared rosiglitazone and reported a yield of 48% for the coupling reaction of 2-(N-methyl-N-(2-pyridyl)amino)ethanol with 4-fluorobenzaldehyde in the presence of N,N-dimethylformamide as a solvent and sodium hydride as a base for the synthesis of 4-[2-(N-methyl-N-(2-pyridyl)amino)ethoxy]benzaldehyde when carried out at room temperature.

Cantello et al. (Bioorganic and Medicinal Chemistry, Vol. 4, No. 10, pp. 1181-1184, 1994) have reported a yield of 72% when the same reaction was carried out at 80° C.

U.S. Pat. No. 6,515,132 B2 describes the reaction of 2-(N-methyl-N-(2-pyridyl)amino)ethanol with 4-fluorobenzaldehyde in the presence of an aprotic polar solvents selected from the group consisting of dimethyl sulphoxide (DMSO), N,N-dimethyl formamide (DMF) and tetrahydrofuran or mixtures thereof and an alkali metal hydroxide or an alkali metal alkoxide as a base at room temperature to obtain 4-[2-(N-methyl-N-(2-pyridyl)amino)ethoxy]benzaldehyde in 88% yield. The said process involves an aprotic polar solvents, an alkali metal alkoxide demands anhydrous conditions, and the presence of water during reaction adversely effects yields and quality of the product.

U.S. Pat. No. 5,741,803 (based on International patent application, publication number WO 94/05659) discloses rosiglitazone maleate, which shows the surprising and advantageous stability and aqueous solubility and provides for significant formulation and bulk handling advantages.

U.S. Pat. No. 6,815,457 (based on International patent application, publication number WO 00/64892) discloses a new polymorphic form of rosiglitazone maleate. The description teaches that the form of rosiglitazone maleate obtained according to the prior art process as described in WO 94/05659 is referred to as “Compound (I)” or “Original Polymorph”.

In the prior art processes for the preparation of pyridyl substituted ethoxy benzyldehyde (ether compound) the presence of even small amount of water in an aromatic aldehyde compound converts it to a corresponding acid and unavoidable amount of the said acid resulted in lower yield.

### SUMMARY OF THE INVENTION

The present invention relates to a novel process for the preparation of a compound represented by the formula (II)

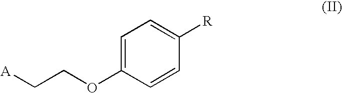

wherein
A is selected from (a) aryl group, (b) a phenyl group optionally substituted by one or two substituents each selected from nitro, halo, C₁-C₄ alkyl, C₁-C₄ alkoxy and hydroxy, (c) a 1- or 2-naphthyl group, (d) pyridyl optionally substituted by lower C₁-C₄ alkyl group, 5- or 6-membered unsaturated heterocyclic ring containing from one to three heteroatoms selected from nitrogen, oxygen or sulfur, 5-ethyl-2-pyridinyl, or N-methyl-N-(2-pyridyl)amino radical,
R is aldehyde, cyano or nitro group.

The present invention provides the process for the preparation of an intermediate compound of the formula (II) which avoids formation of a corresponding aromatic acid from an aldehyde compound in the reaction medium, with high yields and high purity and hence with the low level of the impurity profile (less then 0.1%), which is well under control. The process is simple, industrially easily feasible, economically cheap an environmental friendly process for the preparation of above key intermediate which may be further converted to various thiazolidinedione derivatives such as rosiglitazone, pioglitazone, troglitazone and ciglitazone or pharmaceutically acceptable acid addition salt thereof, preferably to rosiglitazone maleate and rosiglitazone phosphate salt.

The novel process of the invention for the preparation of an intermediate compound of the formula (II) comprising reacting a compound of the formula (III) with a compound of the formula (IV) in a mixture of a non-polar water immiscible solvent and water (two phase system) with an alkali metal hydroxide or an alkali metal carbonate as a base and in the presence of a phase transfer catalyst.

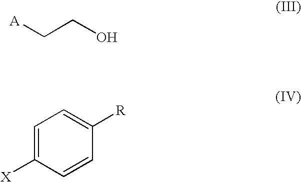

wherein A and R are as defined above, and

X is chlorine, bromine, fluorine or any easy leaving group.

The present invention also provides a process for the preparation of certain thiazolidinedione compounds such as rosiglitazone (maleate or phosphate salt), pioglitazone (HCl), triglitazone and ciglitazone by converting the above key intermediate of formula (II) into the said compounds, useful in the treatment of Type II diabetes.

In the first aspect the present invention relates to the novel process for the preparation of a compound of the formula (V), which comprises reacting a compound of the formula (III), wherein A is N-methyl-N-(2-pyridyl)amino radical, with the compound of formula (IV), wherein X is fluorine and R is aldehyde group, in the mixture of a non-polar water immiscible aromatic hydrocarbon solvent, e.g toluene, and water with an alkali metal hydroxide, e.g. potassium hydroxide, as a base and in the presence of a phase transfer catalyst.

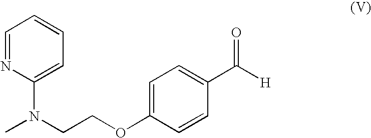

The obtained compound 4-[2-(N-methyl-N-(2-pyridyl)amino)ethoxy]benzaldehyde is the key intermediate for the further synthesis step of rosiglitazone or a pharmaceutically acceptable acid addition salt thereof. The process of the present invention provides an improved process for the preparation of rosiglitazone or a pharmaceutically acceptable acid addition salts thereof.

Rosiglitazone is generic name for 5-[4-[2-(N-methyl-N-(2-pyridyl)amino)ethoxy]benzyl]-thiazolidine-2,4-dione, as illustrated by formula I, which is in the form of its maleate salt contained in commercial drug Avandia®.

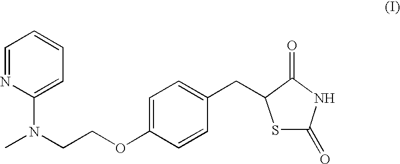

### DETAILED DESCRIPTION OF THE INVENTION

An object of the present invention is to find out a novel process for the preparation of a key intermediate of the formula (II) used in the preparation of certain thiazolidinedione derivatives, such as rosiglitazone and its salts, which would be simple, industrially easily feasible, environmental friendly and which would avoid a non-aqueous medium in order to prevent formation of a corresponding acid from an aromatic aldehyde, e.g. of formula (V), and the degree of purity of thiazolidinedione derivatives, such as rosiglitazone and its salts, prepared according to the present invention would be high with the low level of impurity profile.

We have unexpectedly found that above problem has been solved by the novel process of the invention, which comprises reacting a compound of the formula (III) with a compound of the formula (IV) in a mixture of a non-polar water immiscible solvent and water (two phase system), an alkali metal hydroxide or an alkali metal carbonate as a base and in the presence of a suitable phase transfer agent in catalytic to molar excess amount.

As an alkali metal hydroxide may be used sodium hydroxide, potassium hydroxide, lithium hydroxide or tetra n-butylammonium hydroxide. The preferred alkali metal hydroxide is potassium hydroxide. Potassium carbonate may be used preferably as an alkali metal carbonate.

As a non-polar water immiscible solvent may be used an aromatic hydrocarbon solvents, preferably toluene and xylene, more preferably toluene. Further, as a non-polar water immiscible solvent may be used diethyl ether, ethyl acetate, halogenated hydrocarbon solvents, e.g. methylene chloride.

Any suitable phase transfer catalyst may be used, such as benzyl tri n-butylammonium bromide, benzyltriethylammonium chloride, tetra n-butylammonium bromide, tetra-butylammonium hydrogensulphate or benzyltrimethylammonium chloride.

The process for the preparation of the key intermediate 4-[2-(N-methyl-N-(2-pyridyl)amino)ethoxy]benzaldehyde (V) in the synthesis of rosiglitazone or a pharmaceutically acceptable acid additions salt thereof is represented by the following scheme-1.

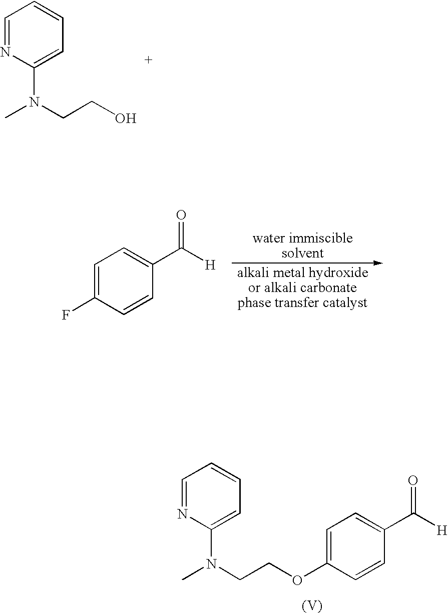

The starting compound for the preparation of rosiglitazone 2-(N-methyl-N-(2-pyridyl)amino)ethanol may be prepared in a manner known per se by reacting 2-chloropyridine with 2-(N-methyl-amino) ethanol.

A further aspect of the invention provides an improved process for the preparation of rosiglitazone or a pharmaceutically acceptable acid addition salt thereof, e.g. its maleate salt or phosphate salt, which comprises:
i.) reacting 2-chloropyridyne with 2-(N-methylamino)ethanol to obtain 2-(N-methyl-N-(2-pyridyl)amino)ethanol;
ii.) reacting 2-(N-methyl-N-(2-pyridyl)amino)ethanol with 4-fluorobenzaldehyde in a mixture of a non-polar water immiscible organic solvent and water with an alkali metal hydroxide or an alkali metal carbonate as a base in the presence of a phase transfer catalyst;
iii.) isolating 4-[2-(N-methyl-N-(2-pyridyl)amino)ethoxy]benzaldehyde;
iv.) contacting 4-[2-(N-methyl-N-(2-pyridyl)amino)ethoxy]benzyldehyde with 2,4-thiazolidinedione in an organic solvent and in the presence of piperidine acetate;
v.) reducing obtained 5-[4-[2-[N-methyl-N-(2-pyridyl)amino)ethoxy]benzylidene-2,4-thiazolidinedione with a dithionite source to rosiglitazone;
vi.) converting rosiglitazone into its pharmaceutically acceptable maleate salt or phosphate salt by reaction with maleic acid or phosphoric acid.

In the step ii.) any suitable non-polar water immiscible solvent may be used as described above, preferably toluene.

In the step ii.) any suitable base may be used as described above, preferably potassium hydroxide.

In the step ii.) any suitable phase transfer catalyst may be used as described above, e.g. tetra n-butylammonium hydrogensulphate, benzyltriethylammonium chloride or tetra n-butylammonium hydroxide. The phase transfer catalyst used in step ii.) ranges in catalytic to molar excess amount.

The reaction temperature of the step ii.) ranges from 20 to 90° C., preferably from 35 to 75° C., more preferably from 49 to 52° C.

The suitable organic solvent used in the step iv.) may be an aromatic hydrocarbon solvent, preferably toluene.

The reducing step v.) is preferably carried out with sodium ditihionite as the dithionite source reducing agent, preferably in a mixture of N,N-dimethylformamide and aqueous solution of potassium carbonate.

The obtained benzylidene-2,4-thiazolidinedione compound from the step iv.) may be purified in an alcoholic medium, e.g. in isopropyl alcohol, in a protic solvent, e.g. N,N-dimethyl formamide, or mixtures thereof.

The reduction of the step v.) may be carried out alternatively by catalytic hydrogenation in a suitable organic solvent in the presence of a catalyst, e.g. Pd/C, or by borohydride reduction, preferably by sodium borohydride, optionally in the presence of a metal catalyst.

Rosiglitazone obtained from the step v.) may be purified in an organic solvent, preferably in an alcohol solvent, e.g. isopropyl alcohol.

The process for preparing rosiglitazone or a pharmaceutically acceptable acid addition salt thereof, preferably its maleate salt or phosphate salt according to the improved method of the present invention involving intermediate compound (V) may be represented by the following scheme-2.

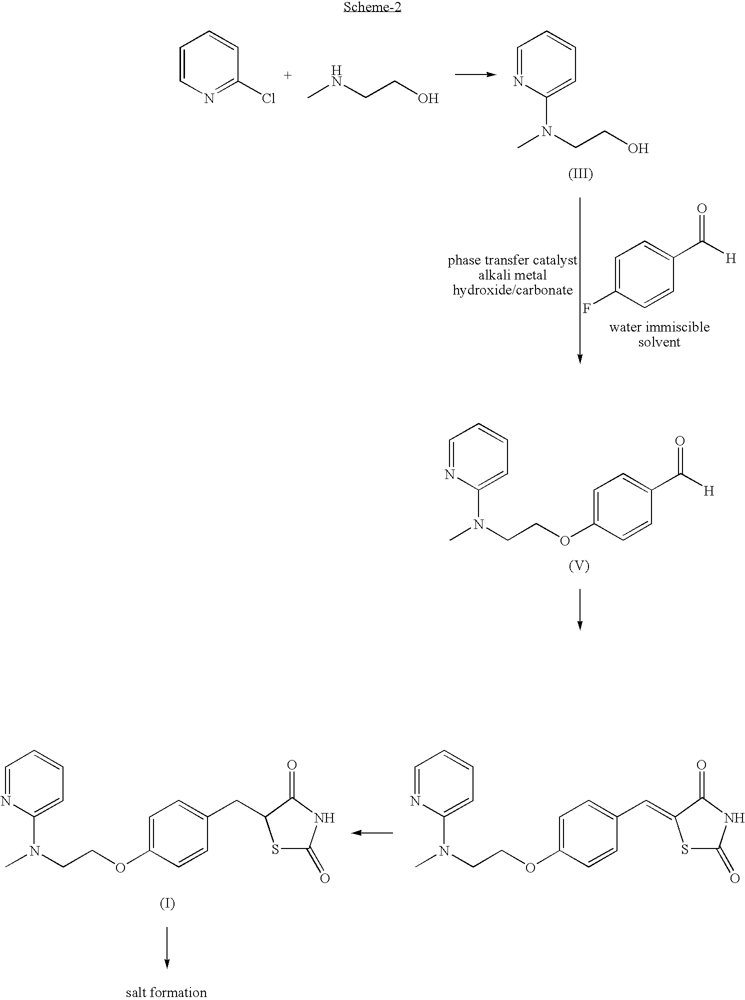

Rosiglitazone maleate prepared according to the present invention is obtained in polymorphic form which corresponds to the polymorphic form of rosiglitazone maleate obtained according to the prior art process of example 1 of WO 94/05659.

Rosiglitazone or a pharmaceutically acceptable acid addition salt thereof obtained by the above described process involving the intermediate compound 4-[2-(N-methyl-N-(2-pyridyl)amino)ethoxy]benzaldehyde (V) obtained according to the process of the present invention may be used in a pharmaceutical compositions by mixing rosiglitazone or a pharmaceutically acceptable acid addition salt thereof with a phisiologically acceptable carrier, excipient, binder, diluent, etc. and may be administered either orally or non-orally. Preferred pharmaceutically acceptable salt is rosiglitazone maleate and rosiglitazone phosphate salt.

The said pharmaceutical compositions may be available in the dosage form including granules, powders, tablets, capsules, syrups, emulsions, suspensions, etc. and non-oral dosage forms, e.g. drip infusions, external application forms. These forms may be manufactured by the techniques known per se conventionally used in pharmaceutical practice.

The invention is illustrated by the following Examples:

### EXAMPLE 1

### Preparation of 2-(N-methyl-N-(2-pyridyl)amino)ethanol

A mixture of 1.5 kg (13.21 mol) of 2-chloropyridine and 12.75 l (158.7 mol) of 2-(N-methylamino) ethanol are stirred for about 22 hours at 120-125° C. Then excess of 2-(N-methylamino) ethanol (about 9.1 l) is distilled off under reduced pressure. 3.0 l of water is added to the oily residue at 25-35° C. and stirred the solution for 30 minutes followed by addition of 3.0 l of toluene and stirred for 30 minutes. The aqueous layer is separated and extracted twice with 3.0 l of toluene. The combined organic layers are washed twice with 1.5 l of water. The organic solvent is evaporated at 50-55° C. under reduced pressure. 1.82 kg (90.55%) of the title compound is obtained as an oil.

### EXAMPLE 2

### Preparation of 4-[2-(N-methyl-N-(2-pyridyl)amino)ethoxy]benzaldehyde

A mixture of 450 ml of toluene, 300 ml of water, 91 g of potassium hydroxide, 50 g of 2-(N-ethyl-N-(2-pyridyl)amino)ethanol, 60 g of 4-fluorobenzaldehyde and 56 g of tetra n-butylammonium hydrogensulphate are heated at 49-52° C. and stirred vigorously for about 20 hours at the same temperature. 300 ml of water is added to the reaction mass stirred for 10-15 minutes and the aqueous layer is separated. The organic layer is washed with 300 ml of water. The combined aqueous layers are extracted with 200 ml of toluene and the layers are separated. The combined toluene layers are extracted with a mixture of 600 ml of water and 40 ml of concentrated hydrochloric acid. The aqueous extract is separated and 80 ml of 12% aqueous ammonium hydroxide solution is added during stirring. Precipitated product is isolated by filtration, washed with water and dried under vacuum to obtain 60.2 g of the title compound as a light yellow colored solid.

### EXAMPLE 2(a)

### Preparation of 4-[2-(N-methyl-N-(2-pyridyl)amino)ethoxy]benzaldehyde

A mixture of 450 ml of toluene, 300 ml of water, 91 g of potassium hydroxide, 50 g of 2-(N-ethyl-N-(2-pyridyl)amino)ethanol, 60 g of 4-fluorobenzaldehyde and 38 g of benzyltriethylammonium chloride are heated at 49-52° C. and stirred for about 20 hours at the same temperature. 300 ml of water is added to the obtained reaction mass and stirred the said mass for 10-15 minutes. The aqueous layer is then separated. The organic layer is washed with 300 ml of water. The combined aqueous layers are extracted with 200 ml of toluene and the layers are separated. The combined toluene layers are extracted with a mixture of 600 ml of water and 40 ml of concentrated hydrochloric acid. The aqueous extract is separated and 80 ml of 12% aqueous ammonium hydroxide solution is added to the said extract during stirring. Precipitated product is isolated by filtration, washed with water and dried under vacuum to obtain 59.8 g of the title compound as a light yellow colored solid.

### EXAMPLE 2(b)

### Preparation of 4-[2-(N-methyl-N-(2-pyridyl)amino)ethoxy]benzaldehyde

A mixture of 250 ml of toluene, 100 ml of water, 45 g of potassium hydroxide, 25 g of 2-(N-ethyl-N-(2-pyridyl)amino)ethanol, 30 g of 4-fluorobenzaldehyde and 53 ml of aqueous solution of tetra n-butylammonium hydroxide are heated at 49-52° C. and stirred vigorously for about 20 hours at the same temperature. 150 ml of water is added to the reaction mass and stirred the said mass for 10-15 minutes. The aqueous layer is then separated. The organic layer is washed with 150 ml of water. The combined aqueous layers are extracted with 100 ml of toluene and layers are separated. The combined toluene layers are extracted with a mixture of 300 ml of water and 20 ml of concentrated hydrochloric acid. The aqueous extract is separated and 40 ml of 12% aqueous ammonium hydroxide solution is added during stirring. Precipitated product is isolated by filtration, washed with water and dried under vacuum to obtain 27.2 g of the title compound as a light yellow colored solid.

### EXAMPLE 3

### Preparation of 5-[4-[2-[N-methyl-N-(2-pyridyl)amino)ethoxy]benzylidene]thiazolidine-2,4-dione

14.4 g of piperidine and 10 g of acetic acid are added to a mixture of 12.0 l of toluene, 2.0 kg (7.8 mol) of 4-[2-(N-methyl-N-(2-pyridyl)amino)ethoxy]benzaldehyde and 1.0 kg (8.78 mol) of 2,4-thiazolidinedione at 25-30° C. The reaction mixture is heated at reflux temperature for 5 hours by removing water azeotropically using Dean Stark apparatus and resulted orange colored reaction mass is allowed to cool to 25-30° C. The separated solid is filtered off and washed with 5.0 l of methanol. The obtained solid is dried at 68-72° C. under reduced pressure for 12 hours to obtain 2.16 kg (75.8%) of the title compound.

### EXAMPLE 3(a)

Purification of 5-[4-[2-[N-methyl-N-(2-pyridyl)]amino)ethoxy]benzylidene]thiazolidine-2,4-dione

2.1 kg of 5-[4-[2-[N-methyl-N-(2-pyridyl)amino)ethoxy]benzylidene]thiazolidine-2,4-dione (obtained from example 3) is dissolved in 9.0 l of N,N-dimethylformamide at 85-90° C. 9.0 l of isopropyl alcohol is added to the solution and allowed to cool to 8-10° C. during stirring for about 1 hour. The separated solid is filtered off and washed with 5 l of isopropyl alcohol. The obtained compound is dried under reduced pressure at 68-72° C. Yield=1.8 kg.

### EXAMPLE 4

### Preparation of 5-[4-[2-[N-methyl-N-(2-pyridyl)amino)ethoxy]benzyl]thiazolidine-2,4-dione (rosiglitazone)

1.0 kg (2.81 mol) of 5-[4-[2-[N-methyl-N-(2-pyridyl)amino)ethoxy]benzylidene]thiazolidine-2,4-dione, 6.0 l of N,N-dimethylformamide are added to a solution of 1.62 kg (11.72 mol) of potassium carbonate in 6.0 l of water and the contents are heated at 69-74° C. during stirring. A solution of 3.8 kg (21.83 mol) of sodium dithionite, 1.2 kg (8.68 mol) of potassium carbonate in 17.0 l of water is added slowly to the reaction mass over the period of 2.5 hours. The reaction mixture is maintained during stirring at 69-74° C. for about 3 hours and is allowed to cool to 50° C. during stirring over the period of 2 hours. The reaction mixture is allowed to cool to 8-10° C. and stirred for 1 hour. The separated solid is then filtered off, washed with 20 l of water and the wet product is dried at 68-72° C. under reduced pressure for 15 hours to obtain 617.0 g (61.35% theoretical) of the title product.

### EXAMPLE 4(a)

### Purification of 5-[4-[2-[N-methyl-N-(2-pyridyl)amino)ethoxy]benzyl]thiazolidine-2,4-dione

600 g of 5-[4-[2-N-methyl-N-(2-pyridyl)amino)ethoxy]benzyl]thiazolidine-2,4-dione (obtained from Example 4) is dissolved in 15 l of isopropyl alcohol at 80-85° C. The obtained solution is filtered to remove undissolved particles and the filtrate is allowed to cool to 25-30° C. for about 3 hours. The separated solid is filtered off and then washed with 1.0 l of isopropyl alcohol. The obtained compound is dried at 68-72° C. to obtain 560 g of the pure title compound.

### EXAMPLE 5

### Preparation of 5-[4-[2-[N-methyl-N-(2-pyridyl)amino)ethoxy]benzyl]thiazolidine-2,4-dione maleate (rosiglitazone maleate)

A mixture of 200.0 g of 5-[4-[2-[N-methyl-N-(2-pyridyl)amino)ethoxy]benzyl]thiazolidine-2,4-dione, 65.5 g (11.3 mol) of maleic acid and 1850 ml of absolute ethanol is stirred at reflux temperature to obtain a clear solution. The clear solution is allowed to cool during stirring to 3-5° C. maintaining the said temperature for 20 hours. Precipitated product is filtered off under nitrogen atmosphere. The resulted compound is dried at 50° C. under reduced pressure for 20 hours to obtain 224 g (84.5% theoretical) of rosiglitazone maleate (the obtained polymorphic form corresponds to the polymorphic form of rosiglitazone maleate obtained according to the process of example 1 of WO 94/05659).

### EXAMPLE 6

### Preparation of 5-[4-[2-[N-methyl-N-(2-pyridyl)amino)ethoxy]benzyl]thiazolidine-2,4-dione maleate (rosiglitazone maleate)

4.7 g of 5-[4-[2-[N-methyl-N-(2-pyridyl)amino)ethoxy]benzyl]thiazolidine-2,4-dione and 1.68 g of maleic acid are suspended in 40 ml of absolute ethanol. The mixture is heated to reflux temperature to obtain a solution which is filtered through diatomeous earth and the filtrate is allowed to cool to ambient temperature. The obtained suspension is allowed to keep in the fridge at approximately 4° C. for 6 hours. The resulted crystalline product is then filtered off and dried under reduced pressure at 50° C. for 17 hours to obtain 4.45 g of rosiglitazone maleate (the obtained polymorphic form corresponds to the polymorphic form of rosiglitazone maleate obtained according to the process of example 1 of WO 94/05659).

### EXAMPLE 7

### Preparation of 5-[4-[2-[N-methyl-N-(2-pyridyl)amino)ethoxy]benzyl]thiazolidine-2,4-dione maleate (rosiglitazone maleate)

4.7 g of 5-[4-[2-[N-methyl-N-(2-pyridyl)amino)ethoxy]benzyl]thiazolidine-2,4-dione and 1.68 g of maleic acid are suspended in 40 ml of absolute ethanol. The mixture is heated to reflux temperature to obtain a solution which is filtered through diatomaceous earth. Seeds of 5-[4-[2-[N-methyl-N-(2-pyridyl)amino)ethoxy]benzyl]thiazolidine-2,4-dione maleate are added and the suspension is allowed to cool to ambient temperature. The obtained suspension is allowed to keep in the fridge at approximately 4° C. for 6 hours. The resulted crystalline product is then filtered off and dried under reduced pressure at 50° C. for 17 hours to obtain 4.17 g of rosiglitazone maleate (the obtained polymorphic form corresponds to the polymorphic form of rosiglitazone maleate obtained according to the process of example 1 of WO 94/05659).

### EXAMPLE 8

### Preparation of 5-[4-[2-[N-methyl-N-(2-pyridyl)amino)ethoxy]benzyl]thiazolidine-2,4-dione maleate (rosiglitazone maleate)

4.7 g of 5-[4-[2-[N-methyl-N-(2-pyridyl)amino)ethoxy]benzyl]thiazolidine-2,4-dione and 1.68 g of maleic acid are suspended in 40 ml of absolute ethanol. The mixture is heated to reflux temperature to obtain a solution which is filtered. The filtrate is allowed to cool to approximately 45° C. Seeds of 5-[4-[2-[N-methyl-N-(2-pyridyl)amino)ethoxy]benzyl]thiazolidine-2,4-dione maleate are added and the suspension is allowed to cool to ambient temperature. The suspension is then allowed to keep in the fridge for 5.5 hours. The resulted crystalline product is then filtered off and dried under reduced pressure for 17 hours to obtain 5.6 g of rosiglitazone maleate (the obtained polymorphic form corresponds to the polymorphic form of rosiglitazone maleate obtained according to the process of example 1 of WO 94/05659).

### EXAMPLE 9

### Preparation of 5-[4-[2-[N-methyl-N-(2-pyridyl)amino)ethoxy]benzyl]thiazolidine-2,4-dione maleate (rosiglitazone maleate)

A mixture of 4.7 g of 5-[4-[2-[N-methyl-N-(2-pyridyl)amino)ethoxy]benzyl]thiazolidine-2,4-dione and 1.68 g of maleic acid are suspended in 40 ml of absolute ethanol. The mixture is heated to reflux temperature to obtain a solution which is filtered. The filtrate is allowed to cool to ambient temperature. Crystallisation of 5-[4-[2-[N-methyl-N-(2-pyridyl)amino)ethoxy]benzyl]thiazolidine-2,4-dione maleate is observed to start at about 35° C. The suspension is kept in the fridge for 3 hours. The resulted crystalline product is then filtered off and dried under reduced pressure at 50° C. for 16 hours to obtain 5.86 g of rosiglitazone maleate (the obtained polymorphic form corresponds to the polymorphic form of rosiglitazone maleate obtained according to the process of example 1 of WO 94/05659).

### EXAMPLE 10

### Preparation of 5-[4-[2-[N-methyl-N-(2-pyridyl)amino)ethoxy]benzyl]thiazolidine-2,4-dione maleate (rosiglitazone maleate)

A mixture of 4.0 g of 5-[4-[2-[N-methyl-N-(2-pyridyl)amino)ethoxy]benzyl]thiazolidine-2,4-dione and 1.68 g of maleic acid are stirred in 37 ml of absolute ethanol and heated at boiling until a solution is obtained. 0.4 g of charcoal is added and after approximately 5 minutes the hot solution is filtered and the resulted mixture is allowed to cool to ambient temperature. After standing in a refrigerator at approximately 4° C. for 17 hours the product is filtered off and dried under reduced pressure at 50° C. for 20 hours to obtain 3.9 g of rosiglitazone maleate (the obtained polymorphic form corresponds to the polymorphic form of rosiglitazone maleate obtained according to the process of example 1 of WO 94/05659).

### EXAMPLE 11

### Preparation of 5-[4-[2-[N-methyl-N-(2-pyridyl)amino)ethoxy]benzyl]-2,4-thiazolidine-2,4-dione phosphate (rosiglitazone phosphate)

10.0 g of 5-[4-[2-[N-methyl-N-(2-pyridyl)amino)ethoxy]benzyl]thiazolidine-2,4-dione is dissolved in 250 ml of ethanol (96%) near the boiling point. The solution is allowed to cool with gentle stirring to 65° C. and 3.78 ml of 85% H₃PO₄ is added. The solution is then allowed to cool to ambient temperature with gentle stirring with aid of mechanical stirrer. The resulted product is then isolated by filtration, washed in 2 portions with a total of 20 ml of ethanol (96%) and dried under reduced pressure for hours at 40° C. to obtain 12.1 g of rosiglitazone phosphate.

1. A process for the preparation of a compound of the formula (II) 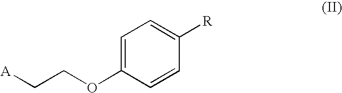
wherein
A is selected from (a) aryl group, (b) a phenyl group optionally substituted by one or two substituents each selected from nitro, halo, C₁-C₄ alkyl, C₁-C₄ alkoxy and hydroxy, (c) a 1- or 2-naphthyl group, (d) pyridyl optionally substituted by C₁-C₄ alkyl group, 5- or 6-membered unsaturated heterocyclic ring containing from one to three heteroatoms selected from nitrogen, oxygen or sulfur, 5-ethyl-2-pyridinyl or N-methyl-N-(2-pyridyl)amino radical,
R is aldehyde, cyano or nitro group, which comprises:
reacting a compound of the formula (III) 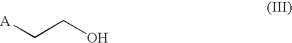
wherein A is as defined above,
with a compound of the formula (IV) 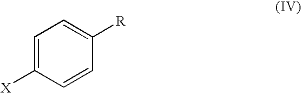
wherein X is chlorine, bromine, fluorine and R is as defined above,
in a mixture of a non-polar water immiscible organic solvent and water with an alkali metal hydroxide or an alkali metal carbonate as a base in the presence of a phase transfer catalyst.

2. The process according to claim 1, wherein the non-polar water immiscible solvent comprises toluene, xylene, diethyl ether, ethyl acetate or halogenated hydrocarbon solvents.

3. The process according to claim 1, wherein the base comprises sodium hydroxide, potassium hydroxide, lithium hydroxide, barium hydroxide, potassium carbonate or sodium carbonate.

4. The process according to claim 1, wherein the alkali metal hydroxide is potassium hydroxide.

5. The process according to claim 1, wherein the phase transfer catalyst comprises benzyl tri n-butylammonium bromide, benzyltriethylammonium chloride, benzyl trimethylammonium chloride, tetra n-butylammonium bromide, tetra n-butylammonium hydrogensulphate, tetramethylammonium chloride or tetra n-butylammonium hydroxide.

6. The process for the preparation of 4-[2-(N-methyl-N-(2-pyridyl)amino)ethoxy]benzaldehyde according to claim 1, which comprises reacting 2-(N-methyl-N-(2-pyridyl)amino)ethanol with 4-fluorobenzaldehyde in the mixture of the non-polar water immiscible organic solvent and water with the alkali metal hydroxide or the alkali metal carbonate as the base in the presence of the phase transfer catalyst.

7. The process according to claim 1, which comprises reacting 2-(N-methyl-N-(2-pyridyl)amino)ethanol with 4-fluorobenzaldehyde in a two phase system of toluene and water with potassium hydroxide in the presence of the phase transfer catalyst.

8. A process for the preparation of rosiglitazone and its pharmaceutically acceptable maleate salt or phosphate salt, which comprises the steps of:
i. reacting 2-chloropyridyne with 2-(N-methylamino)ethanol to obtain 2-(N-methyl-N-(2-pyridyl)amino)ethanol;
ii.) reacting 2-(N-methyl-N-(2-pyridyl)amino)ethanol with 4-fluorobenzaldehyde in a mixture of a non-polar water immiscible organic solvent and water with an alkali metal hydroxide or an alkali metal carbonate as a base in the presence of a phase transfer catalyst;
iii.) isolating 4-[2-(N-methyl-N-(2-pyridyl)amino)ethoxy]benzaldehyde;
iv.) contacting 4-[2-(N-methyl-N-(2-pyridyl)amino)ethoxy]benzaldehyde with 2,4-thiazolidinedione in an organic solvent and in the presence of piperidine acetate;
v.) reducing obtained 5-[4-[2-[N-methyl-N-(2-pyridyl)amino)ethoxy]benzylidene-2,4-thiazolidinedione with a ditionite source to rosiglitazone;
vi.) converting rosiglitazone into its pharmaceutically acceptable maleate salt or phosphate salt, by reaction with maleic acid or phosphoric acid.

9. The process according to claim 8, wherein the non-polar water immiscible solvent is toluene.

10. The process according to claim 8, wherein the base is potassium hydroxide.

11. The process according to claim 8, wherein the phase transfer catalyst is tetra n-butylammonium hydrogensulphate, benzyltriethylammonium chloride or tetra n-butylammonium hydroxide.

12. The process according to claim 8, wherein the organic solvent is toluene.

13. The process according to claim 8, wherein sodium dithionite is the ditionite reducing source.

14. The process according to claim 8, wherein the reducing step is carried out in a mixture of N,N-dimethylformamide and aqueous solution of potassium carbonate.

15. Use of 4-[2-(N-methyl-N-(2-pyridyl)amino)ethoxy]benzaldehyde according to claim 8 for the preparation of rosiglitazone maleate or rosiglitazone phosphate.

16. A pharmaceutical composition comprising a therapeutically effective amount of Type II antidiabetic agent selected from the group consisting of rosiglitazone, pioglitazone, troglitazone or ciglitazone and a pharmaceutically acceptable acid addition salt thereof, prepared according to claim 1, and a pharmaceutically acceptable carrier.

17. The pharmaceutical composition according to claim 16, comprising a therapeutically effective amount of rosiglitazone maleate and a pharmaceutically acceptable carrier.

18. The pharmaceutical composition according to claim 16, comprising a therapeutically effective amount of rosiglitazone phosphate and a pharmaceutically acceptable carrier.
